(19) United States
(12) Patent Application Publication
(10) Pub. No.: US 2007/0173464 A1
(43) Pub. Date: Jul. 26, 2007

(54) Oral ribavirin pharmaceutical compositions

(75) Inventors: Florence Guimberteau, Montussan (FR); Catherine Castan, Orlienas (FR); Remi Meyrueix, Lyon (FR); Gerard Soula, Meyzieu (FR)

Correspondence Address: PATTON BOGGS LLP, 8484 WESTPARK DRIVE, SUITE 900, MCLEAN, VA, 22102, US

(73) Assignee: Flamel Technologies, Venissieux (FR)

(21) Appl. No.: 11/707,034

(22) Filed: Feb. 16, 2007

Related U.S. Application Data
(63) Continuation of application No. 11/449,675, filed on Jun. 9, 2006.
(60) Provisional application No. 60/690,529, filed on Jun. 15, 2005.

(30) Foreign Application Priority Data
Jun. 9, 2005 (FR) PCT/FR05/50434

(51) Int. Cl. A61K 31/7056; A61K 9/22

(52) U.S. Cl. 514/43; 424/468

(57) ABSTRACT

The invention relates to oral pharmaceutical compositions for the prevention and/or the treatment of viral diseases. This invention also addresses methods of prevention and/or treatment of these viral diseases, using these oral compositions. One of the main problems considered in the present invention is to enhance the efficiency of anti-viral treatments, especially against Hepatitis C virus by means of ribavirin, for example in combination with interferon. The oral ribavirin antiviral composition according to the invention increases the bio-absorption time of ribavirin, and thus improves the treatment of patients. Said composition comprises at least one modified release form of ribavirin, the bio-absorption time BAT of which is greater than the bio-absorption time BAT* of a reference* immediate release form of ribavirin administered at the same dose; BAT being preferably comprised between 2 and 15 h and more preferably between 4 and 12 h. Said composition is a reservoir type form or a matrix type form. Said composition is a gastric retentive system or a multiparticulate form.

### RELATED APPLICATIONS

This application claims priority to Provisional Application No. 60/690,529, filed Jun. 15, 2005, and PCT/FR05/050434, filed Jun. 9, 2005.

### BACKGROUND OF THE INVENTION

The invention relates to oral pharmaceutical compositions for the prevention and/or the treatment of viral diseases. This invention also addresses methods of prevention and/or treatment of these viral diseases, using these oral compositions. Viral diseases of particular concern for the invention are notably viral infections of the liver, such as hepatitis C infections.

Inflammation of the liver, or hepatitis, is commonly caused by viruses. Viral hepatitis can be caused by type A (infectious hepatitis), B, C or D viruses. When a person contracts viral hepatitis, the virus invades the liver and causes inflammation of the liver cells. While the body may be able to eliminate the virus on its own, lack of adequate treatment can lead to liver damage such as fibrosis, cirrhosis, liver failure and liver cancer. It is therefore very important to treat hepatitis aggressively.

One current treatment for hepatitis is a combination therapy of pegylated interferon (Peg-Intron or Pegasys) and ribavirin (1-beta-D-ribofuranosyl-1,2,4-thiazole-3-carboxamide). Ribavirin is a purine nucleoside analog with a modified base and a D-ribose sugar, see, e.g., U.S. Pat. No. 3,927,216, which is incorporated in its entirety by reference. Ribavirin inhibits the replication of a wide range of RNA and DNA viruses, including orthomyxo-, paramyxo-, arena-, bunya-, herpes-, adeno-, pox-, and retroviruses. The antiviral mechanism of action of ribavirin is not fully understood but it is believed to relate to alteration of cellular nucleotide pools and inhibition of viral messenger RNA synthesis. One of the benefits of the ribavirin treatment is ribavirin's strong action in viral infected cells.

Ribavirin is currently administered in large dosages, e.g., a dose as large as 1200 mg per day in the form of 4 to 6 capsules per day for treatment of Hepatitis C Virus (HC) infections. Although this dosage is continued daily for a number of months (typically 48 weeks), the resulting efficacy of the treatment of HC, in conjunction either with interferon or long acting pegylated interferon, is however limited. Typically, sustained virologic response is obtained only for 30 to 55% of genotype I patients.

Further, in the treatment of viral infections, it is of major importance to maintain efficient anti-viral concentration at the location of the virus for a sufficient prolonged period of time. In the case of hepatitis, the viral focus is the liver and the portal vein.

Therefore, there is a need for a more efficient treatment of liver viral infections with less side effects that will provide an efficient anti-viral concentration in the liver and portal vein for a prolonged period of time. In this context, one of the main problems considered in the present invention is to enhance the efficiency of anti-viral treatments, especially against HC by means of ribavirin, for example in combination with interferon.

US-A-2005/0019406 discloses sustained release formulations of ribavirin and an excipient based on coated pellets that reduce the dissolution rate of ribavirin in aqueous environment. However, it is our understanding that this patent application does not teach how to enhance the efficiency of anti-viral treatments, especially against HC by means of ribavirin.

WO-A-2005/016370 discloses a ribavirin-based method of treatment of viral infections based on low-dose and/or slow release ribavirin formulation, possibly co-administered with interferon, to provide a clinically effective ribavirin blood level in the portal vein and less than required to provide clinically effective blood level in the systemic circulation. It is our understanding that this patent application does not disclose any particular formulation capable to enhance the efficiency of anti-viral treatments, especially against HC by means of ribavirin.

Thus, a continuing need exists for improved ribavirin compositions for enhancing the efficiency of anti-viral treatments against HC by means of ribavirin.

While not wishing to be constrained by theory, we believe that the sub-optimal efficacy of the treatment with ribavirin and interferon results from the fact that the current treatments of liver viral infections do not maintain the ribavirin concentration in the liver and portal vein, where the virus is located, for a sufficient amount of time.

After oral administration, ribavirin is rapidly bio-absorbed in the very upper part of the small intestine. This narrow window of absorption results in a sharp peak of bio-absorption, one hour after administration, followed by a rapid decline of the absorption rate. After the first pass through the liver, the drug entering the systemic circulation is rapidly distributed in red blood cells, leaving only a minor fraction in plasma. The portal vein and liver are exposed to high concentrations of the drug only during the limited bio-absorption time and the transit time of the drug through liver.

Because the transit time of ribavirin through portal vein and liver is constant, one solution to increase the time that the portal vein and liver are exposed to ribavirin is to increase the bio-absorption time of ribavirin using an appropriate dosage form.

Thus, regarding HC and ribavirin, the inventors have
concentrated their thinkings about the problem of maintaining efficient ribavirin concentration during sufficient prolonged period of time, in the liver and the portal vein;
considered that the transit time of ribavirin through portal vein and liver is fixed at a constant value and cannot be controlled;
and, proposed to increase the time during which portal vein and liver are fed with ribavirin, so as to increase the bio-absorption time of ribavirin using an appropriate dosage form.

Thus, we believe ribavirin-based therapies are improved with a oral pharmaceutical composition that results in increased bio-absorption time, thus exposing, and treating, the portal vein and liver with ribavirin for extended period of time.

The known sustained release formulations that increase the in vitro dissolution time of the drug, particularly the above mentioned ones, do not at all necessary increase the bio-absorption time and the duration of action of a drug such as ribavirin, whose bio-absorption window is narrow. For example, if the release time is too long, the majority of the drug is released when the dosage form is out of the narrow absorption window, resulting in a poor bioavailability without any significant increase of the bio-absorption time. In contrast, if the release time is too short, the bioavailability is acceptable, but the bio-absorption time is short.

Moreover, known sustained release formulations of ribavirin do not necessarily release ribavirin inside the very narrow window of ribavirin bio-absorption. Thus, current sustained released ribavirin formulation do not maintain constant and the therapeutically efficient concentration of ribavirin in the portal vein and in the liver for extended period of time.

Since 1994, the Applicant has developed a microparticle controlled release technology, called Micropump® technology. See, e.g., U.S. Pat. No. 6,022,562, which is incorporated by reference in its entirety. The Micropump® microparticulates are designed to remain in the small intestine for an extended period of time, namely at least about 5 hours, and permit the absorption of the active principle during an extended bio-absorption time. While not wishing to be constrained by any particular theory, we believe the size of the Micropump® microparticulates allows the microparticulates to become trapped in the microvilli of the small intestine. This entrapment by the microvilli allows the microparticulates to remain at the site of greatest bio-absorption of ribavirin, namely the upper part of the small intestine. In U.S. Pat. No. 6,022,562, the active principle can be an antiviral drug, for example acyclovir, whose absorption window is limited to small intestine.

We believe the use of the Micropump® technology, modified as described herein, prolongs the absorption of ribavirin. The microparticles are retained longer in the upper gastrointestinal tract permitting slow release in an environment that appears especially conducive to absorption of ribavirin.

The Micropump® microparticles are 50 to 1000 μm microcapsules of an active principle coated with at least one coating film of specific following composition (by weight of dry matter of the whole coating composition): 50 to 90% ethylcellulose; 2 to 25% polyvinylpyrrolidone; 2 to 20% castor oil and 2 to 20% magnesium stearate. While it is possible to create particles with a narrow size range, the release time of antiviral drugs can be extended by modifying the coat and/or broadening the size range.

When faced with the need to target the very narrow window of bio-absorption to maintain the exposure, and treatment of the portal vein and liver, practitioners will recognize a number of difficulties.

A first difficulty lies in the choice of the most appropriate range of in vitro release profiles.

A second difficulty is to maintain a high ribavirin loading within the dosage form. The slow release of the active principle, namely the ribavirin, can be obtained by coating individually the reservoir-type microparticles by a polymeric membrane a few micrometers thick. After oral administration, the microparticles come in contact with fluids of the gastrointestinal tract and the active principle is slowly released. The driving force for the active principle release is the osmotic pressure of the active principle, directly proportional to its solubility. Ribavirin is a colorless material that has a water solubility of 140 g/liter. Because of its highly soluble nature, sustained release of ribavirin requires thick coating, that reduces the loading rate of the particles. In the case of ribavirin, whose dosage is high, namely up to 3000 mg per day, it is crucial to maximise the drug loading and then to obtain a sustained release of ribavirin with limited amount of coating excipients.

### BRIEF SUMMARY OF THE INVENTION

We propose that the key to increasing the time during which portal vein and liver are exposed to and treated with ribavirin is the fine tuning the ribavirin release time in the gastro-intestinal fluids to simultaneously increase the bio-absorption time and to maintain the bioavailability of the ribavirin at an acceptable level.

Thus, the present invention relates to an oral antiviral drug composition for increasing the bio-absorption time of antiviral drugs, and thus improving the treatment of the patients against viral infection. The improvement of said composition is that it comprises at least one modified release (MR) form of ribavirin the Bio-Absorption Time (BAT) of which is greater than the bio-absorption time BAT* of a reference* immediate release (IR*) form of antiviral drug administered at the same dose. In other words, BAT>BAT*.

### BRIEF DESCRIPTION OF THE DRAWINGS

FIG. 1 illustrates an example of the in vitro release of ribavirin in accordance with one embodiment of the present invention.

### DETAILED DESCRIPTION

In the present invention the increase of the bio-absorption time BAT can be assessed by at least one of the following methods M1 and/or M2.

First Method M1:

The BAT and BAT* values, for the MR and IR* forms, of an antiviral drug are deduced from the plasma concentration profiles over time after dosing of these two forms of antiviral drug: the bio-absorption plasma profile is computed by deconvolution of the mean plasma profile by the input response function of the antiviral drug, as explained in details in “Pharmacokinetics in drug discovery and development, R. D Schoenwald ED., CRC Press, 2002”. The BAT is arbitrarily defined as the time at which 90% of the antiviral drug that will be absorbed is bio-absorbed. Thus, if only 50% of an ingested drug is ultimately absorbed by the organism, the BAT of 90% is achieved when 45% of the ingested drug is absorbed by the organism.

Second Method M2:

The increase of the bio-absorption time is reflected by a flat plasma profile after a single oral dose. The flatness of the plasma profile is measured by the peak and trough ratio, R, which is defmed in the present disclosure as the ratio of the mean plasma concentration at peak, Cmax, divided by the mean plasma concentration 24 h after dosing, C24. In the present invention, an increase of the bio-absorption time will be established if the peak and trough ratio, R, of the formulation according the present invention is less than the peak and trough ratio, R*, of a reference* immediate-release form of the antiviral drug containing the same dose of the antiviral drug administrated in the same conditions. So, when R<R*, then BAT>BAT*.

The oral antiviral drug composition according to the invention can be characterized by the plasma concentration profile, obtained in a reference clinical study in which the oral antiviral drug composition is administered orally, in a single administration, to a sample of N human individuals, preferably N≧20 or 30 human individuals. The individual plasma drug concentration curve as a function of time after dosing is then plotted for each of the patients, from which the individual (i) pharmacokinetic parameters, such as the time Ti,max when the plasma concentration reaches its maximum and the value of this maximum concentration Ci,max, are drawn. Based on these individual parameters, those skilled in the art conventionally calculate the mean values of these parameters and their standard deviations. Further details regarding the discussion of these parameters can be found in Pharmacokinetics and Pharmacodynamic Data Analysis 3rd ed., J. Gabrelsson et al., Kristianstads Bocktryckeri AB, Sweden, 2000.

The experimental conditions of the reference clinical test may for example be the following: administration of the form (gelatin capsule or tablet or suspension) once a day, one hour before breakfast, to 20 normal human volunteers in the course of a cross trial study. The plasma concentrations of antiviral drug are measured at times: 0, 0.25, 0.5, 0.75, 1, 1.5, 2, 3, 4, 6, 8, 10, 12, 16, 18, 24, 36, 48 h post-administration. This clinical test defines the invention by virtue of the pharmacokinetic properties obtained specifically under the conditions of the test. Nevertheless, the invention is not limited to an implementation under the conditions of this reference clinical test.

The term “modified release” denotes, in the present disclosure, a prolonged or sustained release and/or a delayed release and/or a pulsed release of antiviral drug by an oral pharmaceutical composition. Such a modified release oral pharmaceutical composition may, for example, comprise an immediate-release phase and a slow-release phase. Modified-release oral compositions are well known in this field; see, for example, Remington: The Science and practice of pharmacy”, 19th edition, Mack Publishing Co. Pennsylvania, USA. The modified release may in particular be a prolonged and/or delayed release.

“Immediate release form” is intended to denote, in the present disclosure: a form in which most of the amount of the antiviral drug contained therein is released, at pH 6.8 and under SINK conditions in an in vitro dissolution test, in a relatively brief period of time; for example, at least 70% of the antiviral drug is preferably released in 60 min, preferably in 45 min and more preferably in 30 min; or a form in which most of the amount of the antiviral drug contained therein is released in vivo in a relatively brief period of time; for example, at least 70% of the antiviral drug is preferably released in 60 min, preferably in 45 min and more preferably in 30 min, after oral ingestion.

All the dissolution profiles to which reference is made in the present disclosure are determined according to the indications of the European Pharmacopoeia, 4th edition, entitled: “Dissolution test for solid oral forms”: type II dissolutest performed under SINK conditions, at 37° C., and with agitation of 100 rpm.

The bio-absorption time of the oral antiviral drug composition according to the invention is, for example, preferably comprised between 2 and 15 h, and more preferably between 4 and 12 h.

The antiviral drug can be any drug used to treat viral infection of the liver. For instance, the antiviral drug can be a nucleoside analog. In one particular embodiment, the antiviral drug is ribavirin. The term “ribavirin” as used herein includes ribavirin or any analogue, derivative, pro-drug, such as viramidine and levovirin, or ester or salt of such drugs that is found to have virucidal activity.

Pro-drugs are agents which must undergo chemical or enzymatic transformation to the active or parent drug after administration, so that the metabolic product or parent drug can subsequently exhibit the desired pharmacological response. Ribavirin pro-drugs includes pro-drugs from U.S. Pat. No. 6,815,542, which is incorporated in its entirety by reference. Further, ribavirin analogues include analogues from U.S. Pat. No. 6,815,542, which is incorporated in its entirety by reference. Ribavirin derivatives include derivatives from U.S. Pat. No. 6,846,810, which is incorporated in its entirety by reference.

Further, the antiviral drugs of the instant invention include nucleoside analogues and derivatives. These nucleoside analogues and derivatives include those shown in, for example, Kawana et al., J. Org. Chem., 37:288-91 (1972); U.S. Pat. Nos: 3,864,483; 3,914,414; 3,914,415; 3,966,917; 4,029,884; 6,903,079; and in German patents DE 2417465 and DE 2213180.

Advantageously, the oral antiviral drug composition according to the invention can include different MR forms of the antiviral drug, possibly one or several IR form(s) of the drug, as well as possibly at least one MR and/or IR form of one or more other active principle(s). The active principle can be factor hormones, anti-cancer drugs, anti-inflammatories, anti-thrombotics, interferons, pegylated interferons and mixtures thereof.

The inventors have identified what they believe to be an optimum release profile of ribavirin that leads to an extended bio-absorption time of ribavirin without any drastic decrease of the bioavailability.

The inventors demonstrate, after numerous trials, that optimum release profile of the modified release form of ribavirin could be the release profile, obtained in a dissolutest in which the pH is maintained at a pH=1.4 for 1.5 h then increased to a pH=6.8, such that about 70% of the ribavirin is released over a period of time of between about 1.5 and 16 h, preferably between about 2 and 10 h, more preferably between 3 and 8 h.

Thus, whether BAT>BAT* or not, the present invention also refers to an oral ribavirin composition wherein the modified release form of ribavirin has a release profile in a dissolutest in which the pH is maintained at a pH=1.4 for about 1.5 h then increased to a pH=6.8, such that about 70% of the ribavirin is released over a period of time of between about 1.5 and 16 h.

Unless otherwise indicated, use of the term “about” in this disclosure is intended to mean plus or minus 10% of the designated amount; thus, “about 5 to 80%” would mean a range of 4.5-5.5% to 76-84%.

The inventors demonstrate, after numerous trials, that a specific selection of coating compositions allow practitioners to obtain appropriate release profile of the drug with a limited amount of excipients.

The present invention applies principally to the treatment of hepatitis infections and to the use of combinations of interferon and ribavirin. However, the invention also applies to the treatment of any other form of viral infection in which the main tissue damage and the principle site of viral replication is the liver.

A combination of oral ribavirin with interferon α-2b (INF) or peginterferon α-2b is now a standard therapy for treating patients with chronic hepatitis C, particularly with hepatitis C of genotype 1 b and high viral titers. The daily oral dose of ribavirin is currently determined based on the patient's body weight, though approved ribavirin doses vary among countries. In Japan, for example, the dose is 600 mg/d for patients weighing less than 60 kg and 800 mg/d for patients weighing more than 60 kg in a combination therapy with INF. In the United States and Europe, ribavirin is, e.g., administered at a total daily dose of 1000 mg (under 75 kg) or 1200 mg (over 75 kg). Moreover, when ribavirin is combined with peginterferon α-2b, the dose is fixed at 800 mg/d in the United States but is 800 mg/d for patients under 65 kg, 1000 mg/d for patients 65-85 kg, and 1200 mg/d for patients over 85 kg in Europe. More details are found in the following articles, which are incorporated in their entirety by reference: Jen, J., Laughlin, M., Chung, C., Heft, S., Affrime, M. B., Gupta, S. K., Glue, P., and Hajian, G.—Ribavirin dosing in chronic hepatitis C: application of population pharmacokinetic-pharmacodynamic models—Clin Pharmacol Ther, 2002. 72(4): p. 349-61), F. J. Torriani et al., The New England Journal of Medicine, 2004, 351(5), 438-450. Without prejudice of what is above mentioned, it is emphasized that the present invention also covers doses of both ribavirin and/or interferon other than the above given ones.

The interferon(s), possibly combined with the oral ribavirin composition according to the invention, can be co-administered, by any suitable route, with said oral ribavirin and/or can be included in oral ribavirin composition in a therapeutically effective amount. Furthermore, any form of interferon or any derivative thereof may be used in the treatment of the viral infections, including but not limited to interferon alfa or pegylated interferon alfa. Accordingly, the forms of interferon contemplated are those which have been previously shown to have efficacy against hepatitis C or other forms of viral hepatitis. However, the invention also contemplates the use of future forms of interferon including those which may be administered orally in the management of hepatitis.

The term “interferon alpha” as used herein means the family of highly homologous species-specific proteins as defined in U.S. Pat. No. 6,472,373 B1 column 4 line 39 to column 5 line 55.

Other modified interferons, such as the novel genetic fusion protein, Albuferon™ fusion protein (Human Genome Sciences Inc.) are under development. Accordingly, the present invention contemplates the use of ribavirin with any form of interferon or any derivative thereof including the future oral forms of interferon or such derivatives.

According to a first embodiment of the oral ribavirin composition according to the invention, the modified release form of ribavirin is a sustained release form with a release profile at pH 6.8 such that 70% of the ribavirin is released over a period of time, designed hereafter as t(70%), of between about 1.5 and 15 h, preferably between about 2 and 10 h and even more preferably between about 3 and 8 h.

According to a particular sub-embodiment of said first embodiment, the modified release form of ribavirin has an in vitro release profile, in 0.05M potassium dihydrogenophosphate/sodium hydroxide buffer medium at pH 6.8, such that, for any value of time t of between 2 h and t(70%), preferably for any value of time t of between 1 h and t(70%), the % of dissolved (released) ribavirin is greater or equal to 35×t /t(70%).

According to a particular sub-embodiment of said first embodiment, the modified release form can be a gastric retentive system or a multiparticulate form. More preferably, the multiparticulate contains microparticles of the oral antiviral drug composition with a volume mean diameter which is less or equal to about 1000 μm, preferably comprised between about 20 and 800 μm and more preferably comprised between about 50 and 600 μm.

According to a particular sub-embodiment of said first embodiment, the antiviral drug is coated to form a microcapsule where the coating preferably comprises at least one layer to control the modified release of ribavirin, the coating composition of which being as follows:
A. at least one film-forming (co)polymer (A) that is insoluble in the fluids of the gastrointestinal tract;
B. optionally, at least one hydrophilic film-forming (co)polymer (B)
that is insoluble in the fluids of the gastrointestinal tract and
that carries groups that are ionized in the fluids of the gastrointestinal tract,
C. at least one (co)polymer (C) that is soluble in the fluids of the gastrointestinal tract;
D. at least one plasticizer (D);
E. optionally, at least one surfactant and/or lubricant (E).

Preferably, the film-forming (co)polymer (A) that is insoluble in the fluids of the gastrointestinal tract is selected from the group of following products: non-water-soluble derivatives of cellulose, preferably ethylcellulose and/or cellulose acetate, polyvinyl acetates, and mixtures thereof.

Preferably, the hydrophilic film-forming (co)polymer (B) that is insoluble in the fluids of the gastrointestinal tract and that carries groups that are ionized in the fluids of the gastrointestinal tract is selected from the following products: water-insoluble charged acrylic derivatives, preferably from (co)polymers of acrylic and methacrylic acid ester carrying at least one quaternary ammonium group, (B) even more preferably comprising at least one copolymer of alkyl (meth)acrylate and of trimethylammonioethyl methacrylate chloride, and more precisely the products sold under the trade marks Eudragit® RS and/or Eudragit RL, e.g. the powders Eudragit® RL PO and/or Eudragit® RS PO and/or the granules Eudragit® RL 100 and/or Eudragit® RS 100 and/or the suspensions and/or solutions of these Eudragit® RL and Eudragit RS, namely, respectively, Eudragit® RL 30D and/or Eudragit® RS 30D and/or Eudragit® RL 12.5 and/or Eudragit® RS 12.5, and mixtures thereof.

Preferably, the (co)polymer (C) that is soluble in the fluids of the gastrointestinal tract is selected from the group of: nitrogenous (co)polymers, preferably from the group comprising polyacrylamides, poly-N-vinylamides, polyvinylpyrrolidones (PVP) and poly-N-vinyllactams; water-soluble derivatives of cellulose, polyvinyl alcohols (PVAs), polyoxyethylenes (POEs), and mixtures thereof, where polyvinylpyrrolidone is particularly preferred.

Preferably, plasticizer (D) is selected from the group of: cetyl alcohol esters, glycerol and its esters, preferably from the following subgroup: acetylated glycerides, glyceryl monostearate, glyceryl triacetate, glyceryl tributyrate, phthalates, preferably from the following subgroup: dibutyl phthalate, diethyl phthalate, dimethyl phthalate, dioctyl phthalate, citrates, preferably from the following subgroup: acetyl tributyl citrate, acetyltriethyl citrate, tributyl citrate, triethyl citrate, sebacates, preferably from the following subgroup: diethyl sebacate, dibutyl sebacate, adipates, azelates, benzoates, plant oils, fumarates, preferably diethyl fumarate, malates, preferably diethyl malate, oxalates, preferably diethyl oxalate, succinates, preferably dibutyl succinate, butyrates, malonates, preferably diethyl malonate, castor oil (this being particularly preferred), and mixtures thereof.

Preferably, the surfactant and/or lubricant (E) is selected from the group of: anionic surfactants, preferably from the subgroup of alkali metal or alkaline-earth metal salts of fatty acids, stearic acid and/or oleic acid being preferred, and/or nonionic surfactants, preferably from the following subgroup polyoxyethylenated oils, preferably polyoxyethylenated hydrogenated castor oil, polyoxyethylene-polyoxypropylene copolymers, polyoxyethylenated esters of sorbitan, polyoxyethylenated derivatives of castor oil, stearates, preferably calcium stearate, magnesium stearate, aluminum stearate or zinc stearate, stearyl fumarates, preferably sodium stearyl fumarate, glyceryl behenates, and mixtures thereof.

Even more preferably, the coating composition of the first embodiment comprises:
A. film-forming polymer(s) (A) is (are) present in a proportion of 10 to 90%, preferably 40 to 80% by weight on a dry basis relative to the total mass of the coating composition;
B. optional water-insoluble hydrophilic film-forming polymer(s) (B) is (are) present in a proportion of 10 to 90%, preferably 40 to 80% by weight on a dry basis relative to the total mass of the coating composition;
C. polymer(s) (C) that is (are) soluble in the fluids of the gastrointestinal tract is (are) present in a proportion of 2 to 25, preferably 5 to 20% by weight on a dry basis relative to the total mass of the coating composition;
D. plasticizer(s) (D) is (are) present in a proportion of 2 to 20, preferably of 4 to 15% by weight on a dry basis relative to the total mass of the coating composition;
E. optional surfactant(s) and/or lubricant(s) (E) is (are) present in a proportion of 2 to 20, preferably of 4 to 15% by weight on a dry basis relative to the total mass of the coating composition.

For further details, in particular qualitative and quantitative details, regarding at least some of the constituents of this coating composition, reference will be made, for example, to European patent EP-B-0 709 087 or to PCT applications WO-A-2004/010983 and WO-A-2004/010984, which are incorporated by reference in their entirety.

According to a second embodiment of the invention, the oral antiviral composition according to the invention is a modified release form of the antiviral drug that is a sustained release form with an in vitro dissolution behaviour such that:
the release of ribavirin is controlled by means of two distinct triggering mechanisms, one allowing the release of the active principle(s) after a predetermined period of residence in the stomach and the other mechanism being based on a variation in pH or location in the gastrointestinal tract;
at constant pH 1.4, the dissolution profile comprises a lag phase of less than or equal to 7 h, preferably less than or equal to 5 h, and even more preferably of between 1 and 5 h;
and the change from pH 1.4 to pH 7.0 results in a release phase that begins without any lag time.

More preferably, the modified release form of ribavirin according to this second embodiment has an in vitro dissolution behaviour, measured in an in vitro dissolution test, such that:
less than 20% of the ribavirin is released after 2 h at pH 1.4;

at least 50% by weight of the ribavirin is released after 16 h at pH 1.4.

The modified release form of the second embodiment can be a gastric retentive system or a multiparticulate form. Preferably, the multiparticulate form contains microparticles of the oral antiviral drug composition with a mean volume diameter which is less than about 2000 μm, and preferably between about 50 and 800 μm, and even more preferably between about 100 and 600 μm. The volume mean diameter could be measured, for example, with a particle laser sizer.

In one sub-embodiment of the second embodiment, the antiviral drug formulation is a reservoir type: the coating of the microcapsules with modified release of the antiviral drug comprises a composite material
comprising:
at least one hydrophilic polymer A″ carrying groups that are ionized at neutral pH,
at least one hydrophobic compound B″;
representing a mass fraction (% weight relative to the total mass of the microcapsules) ≦40; and
   their mean diameter is less than 2000 μm, and preferably between 50 and 800 μm, and even more preferably between 100 and 600 μm.

Preferably, the composite material A″B″ for the coating for modified release of the active principle with low solubility is such that:
the B″/A″ weight ratio is between 0.2 and 1.5, preferably between 0.5 and 1.0, and the hydrophobic compound B″ is selected from products that are crystalline in the solid state and that have a melting point M_{pB}≧40° C., preferably M_{pB}≧50° C., and even more preferably 40° C.≦M_{pB}≦90° C.

Preferably, the hydrophilic polymer A″ is chosen from: copolymers of (meth)acrylic acid and of (meth)acrylic acid alkyl ester, and mixtures thereof; cellulose derivatives, preferably cellulose acetates, cellulose phthalates, cellulose succinates and mixtures thereof, and even more preferably hydroxypropylmethylcellulose phthalates, hydroxypropylmethylcellulose acetates, hydroxypropylmethylcellulose succinates and mixtures thereof, and mixtures thereof.

More preferably, the polymers A″ are copolymers of (meth)acrylic acid and of (meth)acrylic acid alkyl (e.g. C₁-C₆ alkyl) esters. These copolymers are, for example, of the type of those sold by the company Röhm Pharma Polymers under the registered trade marks Eudragit®, series L and S (such as, for example, Eudragit® L100, Eudragit® S100, Eudragit® L30 D-55 and Eudragit® L100-55). These copolymers are anionic enteric copolymers that are soluble in aqueous medium at pHs above those encountered in the stomach.

Preferably, the compound B″ is chosen from the group of products below: plant waxes taken on their own or as mixtures with one another; hydrogenated plant oils taken on their own or as mixtures with one another; mono- and/or di- and/or triesters of glycerol and of at least one fatty acid; mixtures of monoesters, of diesters and of triesters of glycerol and of at least one fatty acid; and mixtures thereof.

Even more preferably, the compound B″ is chosen from the group of following products: hydrogenated cottonseed oil, hydrogenated soybean seed oil, hydrogenated palm oil, glyceryl behenate, hydrogenated castor oil, tristearin, tripalmitin, trimyristin, yellow wax, hard fat or fat that is useful as suppository bases, anhydrous milk fats, lanolin, glyceryl palmitostearate, glyceryl stearate, lauryl macrogolglycerides, cetyl alcohol, polyglyceryl diisostearate, diethylene glycol monostearate, ethylene glycol monostearate, omega-3 and any mixture thereof, preferably from the subgroup of following products: hydrogenated cottonseed oil, hydrogenated soybean seed oil, hydrogenated palm oil, glyceryl behenate, hydrogenated castor oil, tristearin, tripalmitin, trimyristin and any mixture thereof.

In practice, and without this being limiting, it is preferable for the compound B″ to be chosen: from the group of products sold under the following trade marks: Dynasan®, Cutina®, Hydrobase®, Dub®, Castorwax®, Croduret®, Compritol®, Sterotex®, Lubritab®, Apifil®, Akofine®, Softtisan®, Hydrocote®, Livopol®, Super Hartolan®, MGLA®, Corona®, Protalan®, Akosoft®, Akosol®, Cremao®, Massupol®, Novata®, Suppocire®, Wecobee®, Witepsol®, Lanolin®, Incromega®, Estaram®, Suppoweiss®, Gelucire®, Precirol®, Emulcire®, Plurol diisostéarique®, Geleol®, Hydrine® and Monthyle®, and mixtures thereof; additives for which the codes are as follows: E 901, E 907, E 903 and mixtures thereof; and, preferably, from the group of products sold under the following trade marks: Dynasan® P60, Dynasan® 114, Dynasan® 116, Dynasan® 118, Cutina® HR, Hydrobase® 66-68, Dub® HPH, Compritol® 888, Sterotex® NF, Sterotex® K, Lubritab® and mixtures thereof.

The modified release form of the oral ribavirin composition according to the invention, can be a reservoir type form and/or a matrix type form.

“Reservoir type form” is intended to denote, in the present disclosure, a form in which the volume of material containing the active principle(s) is entirely coated by at least one film that controls the diffusion release speed of the active principle(s) through the continous film (or membrane) which does not include the active principle(s). This release occurs as a result of the contact of the system with the fluids of the gastro intestinal tract. The active principle(s) containing material is, for example, the active principle(s) in itself, a mixture of pharmaceutical excipients or of a mixture of pharmaceutical excipients with the active principle(s). The reservoir form comprises, for example, a plurality of individually coated microcapsules or a monolithic system such as coated tablet(s), a tablet or any other pharmaceutical form containing a plurality of coated microcapsules.

“Matrix type form” is intended to denote, in the present disclosure, a form in which the ribavirin is dispersed in a solid continous (polymeric) phase (the matrix) which controls the diffusion release speed of the active principle(s). Said matrix can or cannot be erodable. Said matrix consists of pharmaceutically acceptable excipients known by the one skilled in the art.

The matrix type form includes, for instance, a plurality of matrix microgranules (matrix elements) containing the active principle(s). These matrix elements are non coated or partially coated by at least one film. The matrix type form can be also a monolithic system (matrix element), such as a tablet(s) non-entirely coated by a continous film and which does not contain any reservoir form. So, the matrix type form can be, e.g. a tablet containing a plurality of active principle(s) IR granules or active principle(s) SR granules, said granules being dispersed in a polymeric matrix.

The modified release form can be a gastric retentive system or a multiparticulate form.

The gastric retentive system may be a dosage form that swells in the stomach or floats on the gastric juice, and thus remains for prolonged period of time in the stomach.

Polymers suitable for use in the gastric retentive system are those that both swell upon absorption of gastric juice and gradually erode over a time period of hours. Erosion initiates simultaneously with the swelling process, upon contact of the surface of the dosage form with gastric fluid. Suitable polymers for use in the present dosage forms may be linear, branched, dendrimeric, or star polymers. Suitable polymers also include synthetic hydrophilic polymers, semi-synthetic, and naturally occurring hydrophilic polymers. The polymers may be homopolymers or copolymers, where the copolymers may be random copolymers, block copolymers or graft copolymers.

Synthetic hydrophilic polymers include, but are not limited to:
Polyalkylene oxides, particularly poly(ethylene oxide), polyethylene glycol and poly(ethylene oxide)-poly(propylene oxide) copolymers;
Cellulosic polymers;
Acrylic acid and metacrylic acid polymers, copolymers and esters thereof, preferably formed from acrylic acid, methacrylic acid, methyl acrylate, ethyl acrylate, methyl methacrylate; ethyl methacrylate, and copolymers thereof, with each other or with additional acrylate species such as aminoethyl acrylate;
Maleic anhydride copolymers;
Polymaleic acid;
Poly(acrylamides) such as polyacrylamide per se, poly(methacrylamide), poly(dimethylacrylamide), and poly(N-isopropyl-acrylamide);
Poly(olefinic alcohol) such as poly(vinyl alcohol);
Poly(N-vinyl lactams) such as poly(vinyl pyrrolidone), poly(N-vinyl caprolactam), and copolymers thereof;
Polyols such as glycerol, polyglycerol (particularly highly branched polyglycerol), propylene glycol and trimethylene glycol substituted with one or more polyalkylene oxides, e.g., mono-, di-, and tri-polyoxyethylated glycerol, mono- and di-poly-oxyethylated propylene glycol, and mono- and di-polyoxyethylated trimethylene glycol;
Polyoxyethylated sorbitol and polyoxyethylated glucose;
Polyoxazolines, including poly(methyloxazoline) and poly(ethyloxazoline);
Polyvinylamines;
Polyvinylacetates, including polyvinylacetate per se as well as ethylene-vinyl acetate copolymers, polyvinyl acetate phthalate, and the like;
Polyimines, such as polyethyleneneimine;
Starch and starch-based polymers;
Polyurethane hydrogels;
Chitosan;
Polysaccharide gums;
Zein; and
Shellac, ammoniated shellac, shellac-acetyl alcohol, and shellac n-butyl stearate.

According to another embodiment, the present invention also encompasses a particular way of implementation of the multiparticulate form that is a reservoir type form, including a plurality of microcapsules with modified release of ribavirin, these microcapsules individually consisting of a microparticle including some ribavirin and coated with at least one coating for modified release of the ribavirin. This multimicroparticulate form can include, inter alia, microcapsules consisting of coated microparticles containing ribavirin. These microparticles containing ribavirin may, for example, be microparticles of the (pure) crude ribavirin in crystal form, matrix granules of ribavirin with various other ingredients, or alternatively neutral microspheres, for example made of cellulose or sugar, coated with at least one layer comprising ribavirin. The ribavirin microcapsules can also contain one or more active principles, different from ribavirin, that are identical to or different from one another.

According to another embodiment, the oral ribavirin composition according to the invention can also include ribavirin MR microparticles (e.g. microcapsules) and MR microparticles (e.g. microcapsules) of one or more active principles that differ from ribavirin, and identical to or different from one another. More generally, these other active principles, different from ribavirin, can be present in the composition in a MR form, such as a matrix form and/or a reservoir form and/or a multiparticulate form and/or a gastroretentive form. To summarize, the composition according to the invention may be made up of various populations of MR microparticles, these populations differing from one another at least through the nature of the active principle(s) (ribavirin) contained therein and/or through the composition of the coating or of the matrix.

The oral ribavirin composition according to the invention can also comprise IR form(s) of ribavirin, and/or possibly of other active principle(s). In particular, these IR forms can be multimicroparticulate forms, namely noncoated microparticles of active principle(s) (ribavirin) that are of the same type as those used in the preparation of the microcapsules as described above.

As regards the structure of the microcapsules used in the composition according to the above sub-embodiment of the invention, two preferred examples of the structure of the microcapsules are given in detail hereinafter, without any implied limitation.

According to one embodiment, at least some of the microcapsules with modified release of antiviral drug each comprise a microparticle of antiviral drug coated with at least one coating for modified release of the antiviral drug. Preferably, the microparticle of antiviral drug is a granule comprising the antiviral drug and one or more pharmaceutically acceptable excipients.

According to another embodiment, at least some of the microcapsules with modified release of active principle(s) (ribavirin) each comprise:
a neutral core,
at least one active layer comprising the active principle(s) and coating the neutral core, and
at least one coating for modified release of the active principle(s) (ribavirin).

According to one embodiment, the neutral core contains sucrose and/or dextrose and/or lactose. According to another embodiment, the neutral core is a cellulose microsphere.

Preferably, the neutral core has a mean diameter of between 1 and 800 μm, and preferably of between 20 and 500 μm. The active layer may optionally comprise, besides the active principle(s) (ribavirin), one or more pharmaceutically acceptable excipients. For example, this active layer can comprise active principle, at least one swelling agent, at least one binder and at least one surfactant.

According to another embodiment, the coating for modified release of the antiviral drug is free from talc.

For further details regarding the preparation of these microcapsules, in particular in the embodiment with a neutral core coated with at least one active layer comprising active principle(s) and with at least one outer coating for modified release of the active principle(s), reference will be made to the content of PCT application WO-A-FR03/030878, which is incorporated here by reference in its entirety.

It is advantageous for the form made up of a plurality of microcapsules to be administered as a large number of microcapsules (typically 5000-50 000).

Without wishing to be limited, it should nevertheless be emphasized that the oral ribavirin composition according to the invention is particularly advantageous in that it can be provided in the form of a single or twice daily oral dose comprising:
from 5000 to 50 000 microunits containing active principle(s), e.g. ribavirin, or
from 5000 to 50 000 microcapsules with modified release of active principle(s), e.g. ribavirin.

This plurality of microcapsules illustrated by the numerical examples mentioned above constitutes a pharmaceutical form that is perfectly well tolerated by the mammalian organism. These microcapsules are all the more advantageous since the production thereof is carried out simply and economically according to techniques well known to those skilled in the art, for example the technique of spray coating in a fluidized air bed, wet granulation, compacting, extrusion-spheronization, etc.

Advantageously, the coating of the microcapsules may comprise, besides the essential constituents, other conventional ingredients known to those skilled in the art, such as in particular: pigments or colorants, plasticizers, (for instance dibutyl sebacate), hydrophilic compounds (for instance cellulose and its derivatives or polyvinylpyrrolidone and its derivatives and mixtures thereof), fillers, anti-foaming agents.

According to an advantageous variant, the composition of the invention, is characterized in that the microcapsule coating responsible for the modified release of ribavirin consists of a single coating layer or a single coating film. This simplifies their preparation and limits the degree of coating. In quantitative terms, the coating monolayer can represent, for example, at most 40%, preferably at most 30%, by weight of the microcapsules. Such a limited amount of coating makes it possible to produce pharmaceutical units each containing a high dose of active principle, without exceeding a size that is totally unacceptable with regard to swallowing.

Such limited coating levels make it possible to produce pharmaceutical units each containing a high dose of ribavirin, without exceeding a size that is totally unacceptable with regard to swallowing.

According to the invention, practical implementations in which the proportion of ribavirin in the microcapsules (expressed as % by weight on a dry basis relative to the total mass of the microcapsules) is between 5 and 90, preferably between 10 and 60, and even more preferably between 20 and 50, are preferred.

Such an optimization of the drug loading occurs by limiting the amount of coating excipients for the sustained release of ribavirin. The remarkable advantage can result from the selection of a particular combination of coating excipients, for instance, to prepare a coating according to the first embodiment as described above. More preferably, this coating can be an impermeable coating having, e.g. the following composition (in % by weight on a dry basis relative to the total mass of the coating composition):
A: 50 to 90, preferably 40 to 80;
B: 0;
C: 5 to 20;
D: 4 to 15;
E: 0.

According to another notable characteristic resulting from the preparation of the microcapsules, the active principle is deposited onto the core by means of techniques known to those skilled in the art, for example the technique of spray coating in a fluidized air bed, wet granulation, compacting, extrusion-spheronization, etc.

The composition according to the invention may comprise, besides microunits consisting of microcapsules with modified release of ribavirin, microunits of ribavirin other than microcapsules. They could, for example, be microparticles with immediate release of ribavirin and/or of one or more other active principle(s). These immediate-release microparticles are advantageously noncoated and may be of the same type as those used in the preparation of the microcapsules according to the invention.

In addition, all the microunits (microparticles and/or microcapsules) constituting the composition according to the invention may be made up of various populations of microunits, these populations differing from one another at least through the nature of the active principle(s) other than ribavirin contained in these microunits and/or through the amount of ribavirin or of other optional active principle(s) that they contain and/or through the composition of the coating and/or through the fact that they are modified-release or immediate-release populations.

Preferably, the microunits containing active principle(s) with immediate release are noncoated microparticles.

The composition according to the invention can be provided in the form of a sachet of powder, of a powder for multidose suspension to be reconstituted in liquid suspension, of a tablet or of a gelatin capsule. The tablet can be advantageously an orally dispersible tablet. The gelatin capsules can contain for example microcapsules.

These tablets, gelatin capsules, powders and suspensions may consist of mixtures of the various populations of microunits, and in particular of microcapsules of active principle(s) according to the invention, preferably combining therewith microunits or microparticles with immediate release of active principle(s), e.g. ribavirin, (for example granules).

Moreover, the invention can be directed towards the use of the microcapsules with modified release of the active principle(s) as defined above, and, optionally, of the microunits containing active principle(s) with immediate release, for preparing pharmaceutical or dietetic, microparticulate oral pharmaceutical forms, preferably in the form of tablets, that are advantageously orally dispersible, of powders or of gelatin capsules.

It should be noted that it may be advantageous to mix, in the same gelatin capsule, in the same tablet or the same powder for oral suspension, at least two types of microcapsules with different ribavirin release kinetics, but that are included within the characteristic framework of the invention.

It may also be recalled that it is possible to mix the microcapsules according to the invention with a certain amount of ribavirin that is immediately available in the organism (immediate release).

Advantageously, the composition containing or not the microcapsules with modified release of ribavirin also comprise pharmaceutically acceptable excipients that are conventional and known to those skilled in the art, useful for example for presenting the microcapsules in tablet form. For example, these excipients may in particular be:
tableting agents such as microcrystalline cellulose or mannitol
dyes
disintegrating agents
flow agents such as talc
lubricants, for instance glyceryl behenate
aromas
preserving agents
and mixtures thereof.

When the composition product is in the form of a tablet, it can be coated according to the techniques and formulations known to those skilled in the art for improving its presentation: color, appearance, taste masking, etc.

As regards the dose, the composition according to the invention can advantageously comprise a daily dose comprised between:
20 and 400 mg;
50 and 800 mg;
50 and 1200 mg;
50 and 2000 mg;
or 50 and 3000 mg.

For instance, the daily dose can be chosen in the group of daily doses (mg) comprising: 400, 800, 1200, 2000 and 3000.

The novel ribavirin-based compositions according to the invention are original in terms of their structure, their presentation and their composition and can be administered per os, in particular by means of single or twice daily doses, for example the doses as defmed above.

In other words, the oral ribavirin composition according to the invention is provided in the form of a unit dosage adapted for a single or a twice daily oral administration.

According to another of its aspects, the invention also relates to the microcapsules per se as defined above.

Finally, the invention is also directed towards a method of treating viral infections in a patient comprising administering to said patient a therapeutic amount of the oral ribavirin composition according to the invention.

Preferably, this method comprises co-administering to the patient, a therapeutically effective amount of at least an interferon.

More preferably, this method is a method of treating hepatitis C.

Moreover, the invention is directed towards the use of the oral ribavirin composition as defined above, for preparing pharmaceutical or dietetic, (e.g. microparticulate), oral galenical forms, preferably in the form of tablets, of powders for oral suspension, of stable liquid suspension or of gelatin capsules.

The invention will be explained more thoroughly by means of the examples hereinafter, given only by way of illustration and enabling the invention to be clearly understood and its variants of implementation and/or of use, along with its various advantages, to be revealed.

### EXAMPLES

### Example 1

### Preparation of 200 mg Ribavirin Capsule

Step 1: Layering

720 g of ribavirin and 80 g of hydroxypropylcellulose (Klucel® EF) are dispersed in 1800 g of water. The suspension is then sprayed onto 200 g of cellulose spheres in a Glatt® GPCG1 fluidized air bed equipment.

Step 2: Coating

850.0 g of granules obtained in step 1 are coated with 105 g of ethylcellulose (Ethocel® 20 Premium/Dow), 20 g of povidone (Plasdone® K29/32/ISP), 15 g of castor oil and 10 g of PEG 40-hydrogenated castor oil (Cremophor® RH40/BASF) dissolved in an ethanol/water (70/30% m/m) mixture, in a Glatt® GPCG1 fluidized air bed equipment.

Step 3: Encapsulation

326 mg of microparticles obtained in step 2 are filled in size 1 gelatin capsule. This capsule contains 200 mg of ribavirin and constitutes the final product.

### Example 2

### Preparation of 200 mg Ribavirin Capsule

Step 1: Granulation

900 g of ribavirin and 100 g of hydroxypropylcellulose (Klucel® EF) are mixed in a high shear granulator (Aeromatic PMA1) during 5 minutes. This mix is then granulated by adding 200 g of water. The product is dried at 40° C. in a ventilated oven and shifted on a 500 μm grid. Finally, the fraction between 200 and 500 μm is selected by sieving.

Step 2: Coating

450.0 g of granules obtained in step 1 are coated with 36 g of ethylcellulose (Ethocel® 20 Premium/Dow), 5 g of povidone (Plasdone® K29/32/ISP), 5 g of castor oil and 4 g of Poloxamer 188 (Lutrol F-68/BASF) dissolved in an ethanol/water (70/30% m/m) mixture, in a Glatt® GPCG1 fluidized air bed equipment.

Step 3: Encapsulation

247 mg of microparticles obtained in step 2 are filled in size 2el gelatin capsule. This capsule contains 200 mg of ribavirin and constitutes the final product.

### Example 3

### Preparation of 200 mg Ribavirin Tablet

Step 1: Granulation

920 g of ribavirin and 80 g of hydroxypropylcellulose (Klucel® EF) are mixed in a high shear granulator (Aeromatic PMA1) during 5 minutes. This mix is then granulated by adding 200 g of water. The product is dried at 40° C. in a ventilated oven and shifted on a 500 μm grid. Finally, the fraction between 200 and 500 μm is selected by sieving.

Step 2: Coating

400.0 g of granules obtained in step 1 are coated with 72 g of ethylcellulose (Ethocel® 20 Premium/Dow), 12 g of povidone (Plasdone K29/32/ISP), 10 g of castor oil and 6 g of Poloxamer 188 (Lutrol F-68/BASF) dissolved in an acetone/isopropyl alcohol (60/40% m/m) mixture, in a Glatt® GPCG1 fluidized air bed equipment.

Step 3: Tabletting

271 g of microparticles obtained in step 2 are mixed with 120 g of microcrystalline cellulose (Avicel PH101), 280 g of mannitol (Pearlitol SD200) and 9 g of magnesium stearate in a Turbula mixer.

Tablets of 680 mg are produced from the above blend in a Korsch alternating press. These tablets contains 200 mg of ribavirin and constitutes the final product.

### Example 4

### In Vitro Dissolution Profiles at pH 6.8

The in vitro release kinetics of ribavirin capsules or tablets prepared in examples 1, 2 and 3 have been determined. The dissolution tests are performed in a USP type II apparatus with a paddle speed of 75 rpm and vessels containing 900 ml of pH 6.8 phosphate buffer (0.05M KH₂PO₄/NaOH) maintained at 37±0.5° C.

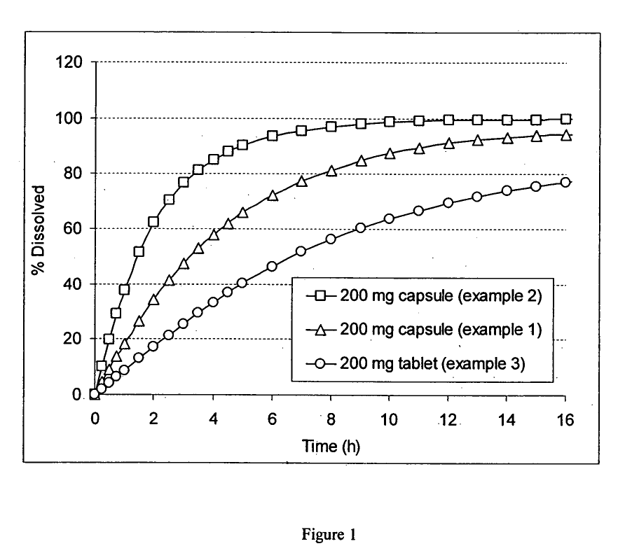

The release profiles of fmal products described in examples 1, 2 and 3 are given in FIG. 1. As shown in FIG. 1, a large range of in vitro release kinetics of ribavirin can be obtained. This permits to prepare products having various in vivo performances.

1. Oral ribavirin antiviral composition for increasing the bio-absorption time of ribavirin, and thus improving the treatment of the patients, said composition comprising at least one modified release form of ribavirin which bio-absorption time BAT is greater than the bio-absorption time BAT* of a reference* immediate release form of ribavirin administered at the same dose;
BAT being preferably comprised between 2 and 15 h and more preferably between 4 and 12 h.

2. Oral ribavirin composition possibly according to claim 1, wherein the modified release form of ribavirin has a release profile, in a dissolutest in which the pH is maintained at a pH=1.4 for 1.5 h then increased to a pH=6.8, such that 70% of the ribavirin is released over a period of time of between 1.5 and 16 h.

3. Oral ribavirin composition according to claim 1 or 2, including a therapeutically effective amount of at least an interferon.

4. Oral ribavirin composition according to claim 2, comprising a modified release form of ribavirin wherein the modified release form of ribavirin is a sustained release form with a release profile at pH 6.8 such that 70% of the ribavirin is released over a period of time, designed as t(70%), of between 1.5 and 15 h, preferably 2 and 10 h and even more preferably between 3 and 8 h.

5. Oral ribavirin composition according to claim 4, wherein the modified release form of ribavirin has an in vitro release profile, in 0.05M potassium dihydrogeno phosphate/sodium hydroxide buffer medium at pH 6.8, such that, for any value of time t of between 2 h and t(70%), preferably for any value of time t of between 1 h and t(70%), the % of dissolved (released) ribavirin is greater or equal to 35×t/t(70%).

6. Oral ribavirin composition according to claim 2, wherein the modified release form of ribavirin is a sustained release form with an in vitro dissolution behaviour such that:
the release of ribavirin is controlled by means of two distinct triggering mechanisms, one being based on a variation in pH and the other allowing the release of the active principle(s) after a predetermined period of residence in the stomach;
at constant pH 1.4 , the dissolution profile comprises a lag phase of less than or equal to 7 h, preferably less than or equal to 5 h, and even more preferably of between 1 and 5 h;,
and the change from pH 1.4 to pH 7.0 results in a release phase that begins without any lag time.

7. Oral ribavirin composition according to claim 6, wherein the modified release form of ribavirin has an in vitro dissolution behaviour, measured in an in vitro dissolution test, such that:
less than 20% of the ribavirin is released after 2 h at pH 1.4;
at least 50% by weight of the ribavirin is released after 16 h at pH 1.4.

8. Oral ribavirin composition according to claim 1 or 2, wherein the modified release form is a reservoir type form.

9. Oral ribavirin composition according to claim 1 or 2, wherein the modified release dosage form is a matrix type form.

10. Oral ribavirin composition according to claim 8 or 9, wherein the modified release form is a gastric retentive system.

11. Oral ribavirin composition according to claim 8 or 9, wherein the modified release form is a multiparticulate form.

12. Oral ribavirin composition according to claim 4 or 5, wherein the microparticles have a mean diameter less or equal to 1000 μm, preferably comprised between 20 and 800 μm and more preferably comprised between 50 and 600 μm.

13. Oral ribavirin composition according to claim 6, wherein the microparticles have a mean diameter less than 2000 μm, and preferably between 50 and 800 μm, and even more preferably between 100 and 600 μm.

14. Oral ribavirin composition according to claim 11, wherein the modified release form is a reservoir type form comprising a plurality of microcapsules with modified release of ribavirin, these microcapsules individually consisting of a microparticle including some ribavirin and coated with at least one coating for modified release of the ribavirin.

15. Oral ribavirin composition according to claim 14, wherein the ribavirin microcapsule coating comprises at least one layer which controls the modified release of ribavirin and the composition of which is as follows:
A) at least one film-forming (co)polymer (A) that is insoluble in the fluids of the gastrointestinal tract;
B) optionally, at least one water-insoluble hydrophilic film-forming (co)polymer
(B) that is insoluble in the fluids of the gastrointestinal tract, carrying groups that are ionized in the fluids of the gastrointestinal tract,
C) at least one (co)polymer (C) that is soluble in the fluids of the gastrointestinal tract;
D) at least one plasticizer (D);
E) optionally, at least one surfactant and/or lubricant (E).

16. Oral ribavirin composition according to claim 15, wherein:
(A) is selected from the group of following products:
non-water-soluble derivatives of cellulose, preferably ethylcellulose and/or cellulose acetate,
polyvinyl acetates,
mixtures thereof;
(B), when it is present, is chosen from water-insoluble charged acrylic derivatives, preferably from (co)polymers of acrylic and methacrylic acid ester carrying at least one quaternary ammonium group, (B) even more preferably comprising at least one copolymer of alkyl (meth)acrylate and of trimethylammonioethyl methacrylate chloride;
(C) is chosen from
nitrogenous (co)polymers, preferably from the group comprising polyacrylamides, poly-N-vinylamides, polyvinylpyrrolidones (PVPs) and poly-N-vinyllactams;
water-soluble derivatives of cellulose,
polyvinyl alcohols (PVAs),
polyoxyethylenes (POEs),
and mixtures thereof;
polyvinylpyrrolidone being particularly preferred;
(D) is chosen from the group comprising:
cetyl alcohol esters,
glycerol and its esters, preferably from the following subgroup: acetylated glycerides, glyceryl monostearate, glyceryl triacetate, glyceryl tributyrate, p1 phthalates, preferably from the following subgroup: dibutyl phthalate, diethyl phthalate, dimethyl phthalate, dioctyl phthalate,
citrates, preferably from the following subgroup: acetyl tributyl citrate, acetyltriethyl citrate, tributyl citrate, triethyl citrate,
sebacates, preferably from the following subgroup: diethyl sebacate, dibutyl sebacate,
adipates,
azelates,
benzoates,
plant oils,
fumarates, preferably diethyl fumarate,
malates, preferably diethyl malate,
oxalates, preferably diethyl oxalate,
succinates, preferably dibutyl succinate,
butyrates,
malonates, preferably diethyl malonate,
castor oil (this being particularly preferred),
and mixtures thereof,
(E) is chosen from the group comprising:
anionic surfactants, preferably from the subgroup of alkali metal or alkaline-earth metal salts of fatty acids, stearic acid and/or oleic acid being preferred,
and/or nonionic surfactants, preferably from the following subgroup:
o polyoxyethylenated oils, preferably polyoxyethylenated hydrogenated castor oil,
polyoxyethylene-polyoxypropylene copolymers,
polyoxyethylenated esters of sorbitan,
polyoxyethylenated derivatives of castor oil,
stearates, preferably calcium stearate, magnesium stearate, aluminum stearate or zinc stearate,
stearyl fumarates, preferably sodium stearyl fumarate,
glyceryl behenates,
and mixtures thereof.

17. Oral ribavirin composition according to claim 16, wherein the composition of the modified-release layer is as follows:
A. the film-forming polymer(s) (A) is (are) present in a proportion of 10 to 90%, preferably from 40 to 80% by weight on a dry basis relative to the total mass of the coating composition;
B. the optional hydrophilic water-insoluble film-forming polymer(s) (B) is (are) present in a proportion of 0 to 90%, preferably 0 to 40% by weight on a dry basis relative to the total mass of the coating composition;
C. the nitrogenous polymer(s) (C) is (are) present in a proportion of 2 to 25, preferably 5 to 20% by weight on a dry basis relative to the total mass of the coating composition;
D. at least one plasticizer (D) is (are) present in a proportion of 2 to 20, preferably of 4 to 15% by weight on a dry basis relative to the total mass of the coating composition;
E. the optional surfactant(s) and/or lubricant(s) (E) is (are) present in a proportion of 2 to 20, preferably of 4 to 15% by weight on a dry basis relative to the total mass of the coating composition.

18. Oral ribavirin composition according to claim 14, wherein the ribavirin microcapsule coating comprises at least one layer which controls the modified release of ribavirin and the composition of which is as follows:
the coating for modified release of the active principle(s) comprises a composite material
including:
at least one hydrophilic polymer A″ carrying groups that are ionized at neutral pH,
at least one hydrophobic compound B″;
representing a mass fraction (% weight relative to the total mass of the microcapsules)<40; and
the microcapsules have a mean diameter of less than 2000 μm.

19. Oral ribavirin composition, according to claim 17, wherein the composite material A″B″ for the coating for modified release of the active principle with low solubility is such that:
the B″/A″ weight ratio is between 0.2 and 1.5, preferably between 0.5 and 1.0,
and the hydrophobic compound B″ is selected from products that are crystalline in the solid state and that have a melting point M_{pB}≧40° C., preferably M_{pB}≧50° C., and even more preferably 40° C. ≦M_{pB}≦90° C.

20. Oral ribavirin composition according to claim 18, wherein the hydrophilic polymer A″ is chosen from:
A″.a copolymers of (meth)acrylic acid and of (meth)acrylic acid alkyl ester, and mixtures thereof;
A″.b cellulose derivatives, preferably cellulose acetates, cellulose phthalates, cellulose succinates and mixtures thereof, and even more preferably hydroxypropylmethylcellulose phthalates, hydroxypropylmethylcellulose acetates, hydroxypropylmethylcellulose succinates and mixtures thereof;
and mixtures thereof.

21. Oral ribavirin composition according to claim 18, wherein the compound B″ is chosen from the group of products below:
B″.a plant waxes taken on their own or as mixtures with one another;
B″.b hydrogenated plant oils taken on their own or as mixtures with one another;
B″.c mono- and/or di- and/or triesters of glycerol and of at least one fatty acid;
B″.d mixtures of monoesters, of diesters and of triesters of glycerol and of at least one fatty acid;
B″.e and mixtures thereof.

22. Oral ribavirin composition according to claim 21, wherein the compound B″ is chosen from the group of following products: hydrogenated cottonseed oil, hydrogenated soybean seed oil, hydrogenated palm oil, glyceryl behenate, hydrogenated castor oil, tristearin, tripalmitin, trimyristin, yellow wax, hard fat or fat that is useful as suppository bases, anhydrous dairy fats, lanolin, glyceryl palmitostearate, glyceryl stearate, lauryl macrogolglycerides, cetyl alcohol, polyglyceryl diisostearate, diethylene glycol monostearate, ethylene glycol monostearate, omega 3 and any mixture thereof,
preferably from the subgroup of following products: hydrogenated cottonseed oil, hydrogenated soybean seed oil, hydrogenated palm oil, glyceryl behenate, hydrogenated castor oil, tristearin, tripalmitin, trimyristin and any mixture thereof.

23. Oral ribavirin composition according to claim 22, wherein the compound B″ is chosen:
from the group of products sold under the following trade marks: Dynasan®, Cutina®, Hydrobase®, Dub®, Castorwax®, Croduret®, Compritol®, Sterotex®, Lubritab®, Apifil®, Akofine®, Softtisan®, Hydrocote®, Livopol®, Super Hartolan®, MGLA®, Corona®, Protalan®, Akosoft®, Akosol®, Cremao®, Massupol®, Novata®, Suppocire®, Wecobee®, Witepsol®, Lanolin®, Incromega®, Estaram®, Suppoweiss®, Gelucire®, Precirol®, Emulcire®, Plurol diisostéarique®, Geleol®, Hydrine® and Monthyle®, and mixtures thereof;
and also from the group of additives for which the codes are as follows: E 901, E 907, E 903 and mixtures thereof;
and, preferably, from the group of products sold under the following trade marks: Dynasan® P60, Dynasan® 114, Dynasan® 116, Dynasan® 118, Cutina® HR, Hydrobase® 66-68, Dub® HPH, Compritol® 888®, Sterotex® NF, Sterotex® K, Lubritab® and mixtures thereof.

24. Oral ribavirin composition according to claim 1 or 2, wherein the daily dose of ribavirin is comprised between:
20 and 400 mg.
50 and 800 mg
50 and 1200 mg.
50 and 2000 mg
or 50 and 3000 mg.

25. Oral ribavirin composition according to claim 24, provided in the form of a unit dosage adapted for a single or a twice daily oral administration.

26. Oral ribavirin composition according to claim 1 or 2, provided in the form of a sachet of powder, of a powder for multidose suspension to be reconstituted in liquid suspension, of a tablet or of a gelatin capsule.

27. A method of treating viral infections in a patient comprising administering to said patient a therapeutic amount of the oral ribavirin composition according to claim 1 or 2.

28. A method according to claim 27, further comprising co-administering to the patient a therapeutically effective amount of at least an interferon.

29. A method according to claim 28, wherein the viral infection is hepatitis C.

30. Use of the oral ribavirin composition according to claim 1 or 2 for preparing pharmaceutical or dietetic, (e.g. microparticulate), oral galenical forms, preferably in the form of tablets, of powders for oral suspension, of stable liquid suspensions or of gelatin capsules.

31. An oral pharmaceutical formulation that comprises at least one antiviral drug that has an in vivo bio-absorption time between about 2 and 15 h, and said bio-absorption time is greater than the bio-absorption time of a reference immediate release form of the antiviral drug administered at the same dose.

32. The oral pharmaceutical formulation that comprises at least one antiviral drug having an in vitro release profile such that about 70% of the antiviral drug is released over a period of time of between about 1.5 and 16 h when the pH is maintained at about pH 1.4 for 1.5 h and then increased to about pH 6.8.

33. The oral pharmaceutical formulation according to claim 31, further comprising an immediate release form of said antiviral drug.

34. The oral pharmaceutical formulation according to claim 31, wherein said formulation further comprises pegylated interferon.

35. The oral pharmaceutical formulation according to claim 31, wherein said antiviral drug comprises a nucleoside analog.

36. The oral pharmaceutical formulation according to claim 31, wherein said antiviral drug comprises ribavirin.

37. The oral pharmaceutical formulation according to claim 31, wherein said antiviral drug has a release profile such that at pH 6.8 about 70% of the ribavirin is released between about 1.5 and 15 h.

38. The oral pharmaceutical formulation according to claim 31, wherein said antiviral drug has a release profile such that at pH 6.8 about 70% of the ribavirin is released between about 2 and 10 h.

39. The oral pharmaceutical formulation according to claim 31, wherein said antiviral drug has a release profile such that at pH 6.8 about 70% of the ribavirin is released between about 3 and 8 h.

40. The oral pharmaceutical formulation according to claim 31, wherein said modified release form is selected from the group of: reservoir type form, matrix type form and gastric retentive form.

41. The oral pharmaceutical formulation according to claim 31, wherein said modified release form comprises microparticles of at least one size.

42. The oral pharmaceutical formulation according to claim 41, wherein the diameter of said microparticles is less than or equal to about 1000 μm.

43. The oral pharmaceutical formulation according to claim 41, wherein the diameter of said microparticles is between about 20 and 800 μm.

44. The oral pharmaceutical formulation according to claim 41, wherein the diameter of said microparticles is between about 50 and 600 μm.

45. The oral pharmaceutical formulation according to claim 41, wherein said modified release form is a reservoir type form which comprises a plurality of microparticles, wherein at least one microparticle is coated to form microcapsules.

46. The oral pharmaceutical formulation according to claim 45, where the coating composition of said microcapsules comprises:
(i) at least one film-forming co-polymer that is relatively insoluble in the fluids of the gastrointestinal tract;
(ii) at least one co-polymer that is relatively soluble in the fluids of the gastrointestinal tract; and
(iii) at least one plasticizer.

47. The oral pharmaceutical formulation according to claim 46, wherein said coating composition further comprises: (iv) at least one water-insoluble hydrophilic film-forming co-polymer that is relatively insoluble in the fluids of the gastrointestinal tract and carries groups that are ionized in the fluids of the gastrointestinal tract.

48. The oral pharmaceutical formulation according to claim 46, wherein said coating composition further comprises:
(v) at least one surfactant.

49. The oral pharmaceutical formulation according to claim 46, wherein said coating composition further comprises
(vi) at least one lubricant.

50. An oral pharmaceutical formulation that comprises at least one antiviral drug in a sustained release form, said antiviral drug having a mean diameter of about 20 to 1000 μM.

51. The oral pharmaceutical formulation according to claim 50, wherein said antiviral drug in a sustained release form has a release profile such that about 70% of the antiviral drug is released over a period of time of between about 1.5 and 16 h when the pH is maintained at about pH 1.4 for 1.5 h and then increased to about pH 6.8.

52. The oral pharmaceutical formulation according to claim 50, wherein said antiviral drug in a sustained release form has a release profile such that about 70% of the antiviral drug is released over a period of time of between about 1.5 and 15 h when the pH is maintained at about pH 6.8.

53. The oral pharmaceutical formulation according to claim 50, wherein said antiviral drug in a sustained release form has a release profile such that about 70% of the antiviral drug is released over a period of time of between about 2 and 10 h when the pH is maintained at about pH 6.8.

54. The oral pharmaceutical formulation according to claim 50, wherein said antiviral drug in a sustained release form has a release profile such that about 70% of the antiviral drug is released over a period of time of between about 3 and 8 h when the pH is maintained at about pH 6.8.

55. The oral pharmaceutical formulation according to claim 50, wherein said antiviral drug comprises a nucleoside analog.

56. The oral pharmaceutical formulation according to claim 50, wherein said antiviral drug comprises ribavirin.

57. The oral pharmaceutical formulation according to claim 50, further comprising a pegylated interferon.

58. The oral pharmaceutical formulation according to claim 50, wherein said antiviral drug is coated so as to form microcapsules.

59. The oral pharmaceutical formulation according to claim 58, wherein the coating composition of said microcapsules comprises:
(i) at least one film-forming co-polymer that is relatively insoluble in the fluids of the gastrointestinal tract;
(ii) at least one co-polymer that is relatively soluble in the fluids of the gastrointestinal tract; and
(iii) at least one plasticizer.

60. The oral pharmaceutical formulation according to claim 59, wherein said coating composition further comprises:
(iv) at least one water-insoluble hydrophilic film-forming co-polymer that is relatively insoluble in the fluids of the gastrointestinal tract and carries groups that are ionized in the fluids of the gastrointestinal tract.

61. The oral pharmaceutical formulation according to claim 59, wherein said coating composition further comprises:
(v) at least one surfactant.

62. The oral pharmaceutical formulation according to claim 59, wherein said coating composition further comprises
(vi) at least one lubricant.

63. The oral pharmaceutical formulation according to claim 59, wherein:
(i) said film-forming co-polymer that is relatively insoluble in the fluids of the gastrointestinal tract is chosen from the group consisting of: non-water-soluble derivatives of cellulose, polyvinyl acetates, and mixtures thereof;
(ii) said co-polymer that is relatively insoluble in the fluids of the gastrointestinal tract is chosen from the group consisting of: nitrogenous co-polymers, water-soluble derivatives of cellulose, polyvinyl alcohols, polyoxyethylenes, and mixtures thereof, and
(iii) said plasticizer is chosen from the group consisting of: cetyl alcohol esters, glycerol, glycerol esters, phthalates, citrates, sebacates, adipates, azelates, benzoates, plant oils, fumarates, malates, oxalates, succinates, butyrates, malonates, castor oil, and mixtures thereof.

64. The oral pharmaceutical formulation according to claim 59, wherein said coating composition further comprises:
(iv) at least one water-insoluble hydrophilic film-forming co-polymer that is relatively insoluble in the fluids of the gastrointestinal tract, carries groups that are ionized in the fluids of the gastrointestinal tract, and is a relatively water-insoluble, charged acrylic derivative.

65. The oral pharmaceutical formulation according to claim 59, wherein:
(i) said film-forming co-polymer that is relatively insoluble in the fluids of the gastrointestinal tract is present in a proportion of about 10 to 90% by weight on a dry basis relative to the total mass of the coating composition.

66. The oral pharmaceutical formulation according to claim 59, wherein:
(i) said film-forming co-polymer that is relatively insoluble in the fluids of the gastrointestinal tract is present in a proportion of about 40 to 80% by weight on a dry basis relative to the total mass of the coating composition.

67. The oral pharmaceutical formulation according to claim 59, wherein:
(ii) said co-polymer that is relatively insoluble in the fluids of the gastrointestinal tract is present in a proportion of about 2 to 25% by weight on a dry basis relative to the total mass of the coating composition.

68. The oral pharmaceutical formulation according to claim 59, wherein:
(iii) said co-polymer that is relatively insoluble in the fluids of the gastrointestinal tract is present in a proportion of about 5 to 20% by weight on a dry basis relative to the total mass of the coating composition.

69. The oral pharmaceutical formulation according to claim 59, wherein:
(iii) said plasticizer is present in a proportion of about 2 to 20% by weight on a dry basis relative to the total mass of the coating composition.

70. The oral pharmaceutical formulation according to claim 59, wherein: (iii) said plasticizer is present in a proportion of about 4 to 15% by weight on a dry basis relative to the total mass of the coating composition.

71. The oral pharmaceutical formulation according to claim 60, wherein:
(iv) at least one water-insoluble hydrophilic film-forming co-polymer that is relatively insoluble in the fluids of the gastrointestinal tract is present in a proportion of about 0 to 90% by weight on a dry basis relative to the total mass of the coating composition.

72. The oral pharmaceutical formulation according to claim 60, wherein:
(iv) at least one water-insoluble hydrophilic film-forming co-polymer that is relatively insoluble in the fluids of the gastrointestinal tract is present in a proportion of about 0 to 40% by weight on a dry basis relative to the total mass of the coating composition.

73. The oral pharmaceutical formulation according to claim 61, wherein:
(v) at least one surfactant is present in a proportion of about 2 to 20% by weight on a dry basis relative to the total mass of the coating composition.

74. The oral pharmaceutical formulation according to claim 61, wherein:
(v) at least one surfactant is present in a proportion of about 4 to 15% by weight on a dry basis relative to the total mass of the coating composition.

75. The oral pharmaceutical formulation according to claim 62, wherein:
(vi) at least one lubricant is present in a proportion of about 2 to 20% by weight on a dry basis relative to the total mass of the coating composition.

76. The oral pharmaceutical formulation according to claim 62, wherein:
(vi) at least one lubricant is present in a proportion of about 4 to 15% by weight on a dry basis relative to the total mass of the coating composition.

77. The oral pharmaceutical formulation according to claim 51, wherein the dose of said antiviral drug is between about 50 and 400 mg a day.

78. The oral pharmaceutical formulation according to claim 51, wherein the dose of said antiviral drug is between about 50 and 800 mg a day.

79. The oral pharmaceutical formulation according to claim 51, wherein the dose of said antiviral drug is between about 50 and 1200 mg a day.

80. The oral pharmaceutical formulation according to claim 51, wherein the dose of said antiviral drug is between about 50 and 2000 mg a day.

81. The oral pharmaceutical formulation according to claim 51, wherein the dose of said antiviral drug is between about 50 and 3000 mg a day.

82. The oral pharmaceutical formulation according to claim 51, wherein the formulation is administered once a day.

83. The oral pharmaceutical formulation according to claim 51, wherein the formulation is administered twice a day.

84. The oral pharmaceutical formulation according to claim 51, wherein said formulation is in a form chosen from the group comprising: sachet of powder, powder for multidose suspension to be reconstituted in liquid suspension, tablet, and a gelatin capsule.

85. The oral pharmaceutical formulation according to claim 51 further comprising an active principle.

86. The oral pharmaceutical formulation according to claim 85, wherein said active principle is selected from the group of: growth factor hormones, anti-cancer drugs, anti-inflammatories, anti-thrombotics, interferons, and mixtures thereof.

87. The oral pharmaceutical formulation according to claim 50, wherein said modified release form is a gastric retentive system form.

88. The oral pharmaceutical formulation according to claim 87, wherein said gastric retentive form comprising polymers, said polymers are chosen from the group comprising of: synthetic hydrophilic polymers, semi-synthetic polymers, naturally occurring hydrophilic polymers, and a mix thereof.

89. An oral pharmaceutical formulation comprising at least one antiviral drug, wherein said antiviral drug is released by at least two mechanisms, wherein first said mechanism is antiviral drug release after a predetermined period of residence in the stomach.

90. The oral pharmaceutical formulation according to claim 89, further comprising a second mechanism, wherein said second mechanism is antiviral drug release dependent upon location of the formulation in the gastro-intestinal tract.

91. The oral pharmaceutical formulation according to claim 89, further comprising a second mechanism, wherein said second mechanism is antiviral drug release upon change in pH.

92. The oral pharmaceutical formulation according to claim 91, wherein said antiviral drug has a release profile such that at about pH 1.4 the antiviral drug is not released for about 7 h and at about pH 7.0 the antiviral drug is released.

93. The oral pharmaceutical formulation according to claim 91, wherein said antiviral drug has a release profile such that at about pH 1.4 the antiviral drug is not released for about 5 h and at about pH 7.0 the antiviral drug is released.

94. The oral pharmaceutical formulation according to claim 91, wherein said antiviral drug has a release profile such that at about pH 1.4 the antiviral drug is not released for about 1 to 5 h and at about pH 7.0 the antiviral drug is released.

95. The oral pharmaceutical formulation according to claim 89, wherein said antiviral drug has a release profile such that about 20% by weight of the antiviral drug is released after about 2 h at about pH 1.4 , and at least about 50% by weight of the antiviral drug is released after 16 h at pH 1.4.

96. The oral pharmaceutical formulation according to claim 89, wherein said antiviral drug comprises a nucleoside analog.

97. The oral pharmaceutical formulation according to claim 96, wherein said antiviral drug comprises ribavirin.

98. The oral pharmaceutical formulation according to claim 89, further comprising a pegylated interferon.

99. The oral pharmaceutical formulation according to claim 89, wherein said antiviral drug is coated so as to form microcapsules having a mean diameter of about 20 to 2000 μm.

100. The oral pharmaceutical formulation according to claim 99, said microcapsules comprising at least one coat with a percent weight relative to the total mass of the microcapsules of less than about 40, wherein the coating composition comprises:
(i) at least one hydrophilic polymer A carrying groups that are ionized at neutral pH, and
(ii) at least one hydrophobic compound B.

101. The oral pharmaceutical formulation according to claim 100 wherein said hydrophilic polymer A is chosen from the group consisting of: copolymers of (meth)acrylic acid, copolymers of (meth)acrylic acid alkyl ester, cellulose derivatives, preferably cellulose acetates, cellulose phthalates, cellulose succinates and mixtures thereof, and even more preferably hydroxypropylmethylcellulose phthalates, hydroxypropylmethylcellulose acetates, hydroxypropylmethylcellulose succinates and mixtures thereof.

102. The oral pharmaceutical formulation according to claim 100 wherein said hydrophobic polymer B is chosen from the group consisting of: plant waxes, hydrogenated plant oils, monoester of glycerol, diesters of glycerol, triesters of glycerol, fatty acid, hydrogenated cottonseed oil, hydrogenated soybean seed oil, hydrogenated palm oil, glyceryl behenate, hydrogenated castor oil, tristearin, tripalmitin, trimyristin, yellow wax, hard fat or fat that is useful as suppository bases, anhydrous dairy fats, lanolin, glyceryl palmitostearate, glyceryl stearate, lauryl macrogolglycerides, cetyl alcohol, polyglyceryl diisostearate, diethylene glycol monostearate, ethylene glycol monostearate, omega 3, Dynasan®, Cutina®, Hydrobase®, Dub®, Castorwax®, Croduret®, Compritol®, Sterotex®, Lubritab®, Apifil®, Akofine®, Softtisan®, Hydrocote®, Livopol®, Super Hartolan®, MGLA®, Corona®, Protalan®, Akosoft®, Akosol®, Cremao®, Massupol®, Novata®, Suppocire®, Wecobee®, Witepsol®, Lanolin®, Incromega®, Estaram®, Suppoweiss®, Gelucire®, Precirol®, Emulcire®, Plurol diisostéarique®, Geleol®, Hydrine® and Monthyle®, E 901 additive, E 907 additive, E 903 additive and mixtures thereof.

103. The oral pharmaceutical formulation according to claim 102 wherein said hydrophobic compound B is crystalline in the solid state and is chosen from the group consisting of: compounds with a melting point of M_{pB}≧40° C., compounds with a melting point of M_{pB}≧50° C., compounds with a melting point of 40° C. ≦M_{pB}≦90° C., and mixtures thereof.

104. The oral pharmaceutical formulation according to claim 102 wherein the weight ratio of said hydrophobic compound B to said hydrophilic compound A is between about 0.2 and 1.5.

105. The oral pharmaceutical formulation according to claim 102 wherein the weight ratio of said hydrophobic compound B to said hydrophilic compound A is between about 0.5 and 1.0.

106. The oral pharmaceutical formulation according to claim 89, wherein the dose of said antiviral drug is between about 50 and 400 mg a day.

107. The oral pharmaceutical formulation according to claim 89, wherein the dose of said antiviral drug is between about 50 and 800 mg a day.

108. The oral pharmaceutical formulation according to claim 89, wherein the dose of said antiviral drug is between about 50 and 1200 mg a day.

109. The oral pharmaceutical formulation according to claim 89, wherein the dose of said antiviral drug is between about 50 and 2000 mg a day.

110. The oral pharmaceutical formulation according to claim 89, wherein the dose of said antiviral drug is between about 50 and 3000 mg a day.

111. The oral pharmaceutical formulation according to claim 89, wherein the formulation is administered once a day.

112. The oral pharmaceutical formulation according to claim 89, wherein the formulation is administered twice a day.

113. The oral pharmaceutical formulation according to claim 89, wherein said formulation is in a form chosen from the group comprising: sachet of powder, powder for multidose suspension to be reconstituted in liquid suspension, tablet, and a gelatin capsule.

114. The oral pharmaceutical formulation according to claim 89 further comprising an active principle.

115. The oral pharmaceutical formulation according to claim 114, wherein said active principle is selected from the group of: growth factor hormones, anti-cancer drugs, anti-inflammatories, anti-thrombotics, interferons, and mixtures thereof.

116. The oral pharmaceutical formulation according to claim 89, wherein said modified release form is a gastric retentive system form.

117. The oral pharmaceutical formulation according to claim 116, wherein said gastric retentive form comprises polymers, said polymers being chosen from the group comprising of: synthetic hydrophilic polymers, semi-synthetic polymers, naturally occurring hydrophilic polymers, and a mix thereof.
